(19) United States
(12) Patent Application Publication
(10) Pub. No.: US 2025/0370129 A1
(43) Pub. Date: Dec. 4, 2025

(54) SYSTEM AND METHOD FOR ULTRASOUND IMAGING

(71) Applicant: CORPORATION DE L’ÉCOLE POLYTECHNIQUE DE MONTRÉAL, Montréal (CA)

(72) Inventors: Jean PROVOST, Montréal (CA); Vincent PERROT, Montréal (CA); Olivier CARON-GRENIER, - - (CA); Jonathan POREE, Montréal (CA)

(21) Appl. No.: 18/876,453

(22) PCT Filed: Jun. 21, 2023
(86) PCT No.: PCT/CA2023/050865

§ 371 Date: Dec. 18, 2024

Related U.S. Application Data
(60) Provisional application No. 63/353,951, filed on Jun. 21, 2022.

(51) Int. Cl. G01S 15/89; G01S 7/52

(52) U.S. Cl. CPC G01S 15/8977; G01S 7/52028; G01S 15/8913; G01S 15/8915

(57) ABSTRACT

There is provided a system for ultrasound imaging comprising at least one ultrasonic transmitter configured to transmit at least one ultrasonic wave towards at least one target, an ergodic relay coupled to the at least one target, the ergodic relay configured for receiving at least one ultrasonic wave backscattered by the at least one target and for applying a temporal signature, as a function of a location of the at least one target relative to the ergodic relay, to the at least one backscattered wave to generate at least one output signal, and an ultrasonic receiver coupled to the ergodic relay and configured to receive the at least one output signal therefrom, the at least one output signal used to reconstruct at least one image of the at least one target.

### CROSS-REFERENCE TO RELATED APPLICATIONS

The present application claims priority on U.S. Patent Application No. 63/353,951 filed Jun. 21, 2022, the entire contents of which are incorporated herein by reference.

### FIELD

The improvements generally relate to the field of ultrasound imaging, and more specifically to ultrasound imaging systems and methods including an ergodic relay.

### BACKGROUND

Ultrasonic probes currently use hundreds or even thousands of piezoelectric elements which emit and receive ultrasound signals to form two-dimensional (2D) or three-dimensional (3D) images/volumes of an insonified medium. Typically, the raw received ultrasound signals are converted into an output image or volume, by delaying and summing the received signals. The chosen delays in effect select a pixel to reconstruct based on the time-of-flight. To amplify the signal coming from a certain region, ultrasound waves can be emitted at different intervals during transmission, so that a waveform is created that follows a straight beam over an entire line. By changing this input delay, the position of these lines can be changed in turn and an image is created by “scanning” the medium. The complex electrical connections and the amount of data resulting from the large number of piezoelectric elements are a fundamental limitation for low-cost ultrasound imaging and limit potential clinical application, as well as the available field of view.

Therefore, improvements are needed.

### SUMMARY

In accordance with one aspect, there is provide a system for ultrasound imaging. The system comprises at least one ultrasonic transmitter configured to transmit at least one ultrasonic wave towards at least one target, an ergodic relay coupled to the at least one target, the ergodic relay configured for receiving at least one ultrasonic wave backscattered by the at least one target and for applying a temporal signature, as a function of a location of the at least one target relative to the ergodic relay, to the at least one backscattered wave to generate at least one output signal, and an ultrasonic receiver coupled to the ergodic relay and configured to receive the at least one output signal therefrom, the at least one output signal used to reconstruct at least one image of the at least one target.

In some embodiments, the at least one ultrasonic transmitter is configured to transmit the at least one ultrasonic wave having a given transmission frequency in the at least one target.

In some embodiments, the at least one ultrasonic transmitter comprises an array of ultrasonic transducer elements.

In some embodiments, the ultrasonic receiver comprises a single ultrasonic transducer element.

In some embodiments, the ergodic relay comprises a right-angle prism.

In some embodiments, the system further comprises a computing device communicatively coupled to the ultrasonic receiver, the computing device configured to receive the at least one output signal from the ultrasonic receiver and to reconstruct the at least one image of the at least one target based on the at least one output signal and a dictionary of reception signals.

In some embodiments, the computing device is configured to reconstruct the at least one image of the at least one target as follows:

$$s ( \vec{r} ) = {\sum }_{t} {e}_{\vec{r}} ( t ) \times s ( t )$$

where s(\vec{r}) is the at least one image of the at least one target as reconstructed, s(t) is the at least one output signal, and e_{\vec{r}}(t) is the dictionary of reception signals determined from a unique point reflector located at \vec{r}.

In some embodiments, the computing device is configured to determine the dictionary of reception signals during a calibration procedure in which at least one strong scatterer is imaged with an ultrasound probe to generate the reception signals and at least one reference image associated therewith.

In some embodiments, the computing device is configured to apply at least one deep learning technique to at least one of reconstruct the at least one image of the at least one target and perform a calibration procedure to determine the dictionary of reception signals.

In some embodiments, the computing device is configured to use a dilated convolution kernels architecture for performing the calibration procedure.

In some embodiments, the ergodic relay is configured for applying the temporal signature to the at least one backscattered wave comprising forming the temporal signature between a time of transmission of the at least one ultrasonic wave to the at least one target and a time of reception of the at least one output signal by the ultrasonic receiver, and encoding a spatial location of the at least one backscattered wave with the temporal signature.

In accordance with another aspect, there is provided a method for ultrasound imaging. The method comprises, at a computing device, causing at least one ultrasonic wave to be transmitted towards at least one target, receiving at least one output signal generated by an ergodic relay coupled to the at least one target, the at least output signal generated by the ergodic relay applying a temporal signature, as a function of a location of the at least one target relative to the ergodic relay, to at least one ultrasonic wave backscattered by the at least one target, reconstructing at least one image of the at least one target based on the at least one output signal and a dictionary of reception signals, and outputting the at least one image as reconstructed.

In some embodiments, the at least one image of the at least one target is reconstructed based on the at least one output signal and the dictionary of reception signals as follows:

$$s ( \vec{r} ) = {\sum }_{t} {e}_{\vec{r}} ( t ) \times s ( t )$$

where s(\vec{r}) is the at least one image of the at least one target as reconstructed, s(t) is the at least one output signal, and e_{\vec{r}}(t) is the dictionary of reception signals determined from a unique point reflector located at.

In some embodiments, the method further comprises performing a calibration procedure for determining the dictionary of reception signals, the calibration procedure comprising imaging at least one strong scatterer with an ultrasound probe to generate the reception signals and at least one reference image associated therewith.

In some embodiments, at least one deep learning technique is used to at least one of reconstruct the at least one image of the at least one target and perform a calibration procedure for determining the dictionary of reception signals.

In some embodiments, at least one harmonic imaging technique is used to at least one of said reconstruct the at least one image of the at least one target and said perform the calibration procedure.

In some embodiments, the at least output signal is generated by the ergodic relay applying the temporal signature to the at least one backscattered wave, comprising forming the temporal signature between a time of transmission of the at least one ultrasonic wave to the at least one target and a time of reception of the at least one output signal, and encoding a spatial location of the at least one backscattered wave with the temporal signature.

In accordance with yet another aspect, there is provided a calibration method for ultrasound imaging. The method comprises causing at least one ultrasonic wave to be transmitted towards at least one strong scatterer, receiving at least one reception signal generated by an ergodic relay coupled to the at least one strong scatterer, associating the at least one reception signal with a position of the at least one strong scatterer, and storing the associated at least one reception signal in a dictionary.

In some embodiments, the at least reception signal is generated by the ergodic relay by applying a temporal signature, as a function of a location of the at least one strong scatterer relative to the ergodic relay, to at least one ultrasonic wave backscattered by the at least one strong scatterer.

In some embodiments, the at least one ultrasonic wave is caused to be transmitted towards the at least one strong scatterer comprising a cloud of microbubbles

Many further features and combinations thereof concerning embodiments described herein will appear to those skilled in the art following a reading of the instant disclosure.

### DESCRIPTION OF THE FIGURES

In the figures,

FIG. 1A is a schematic diagram of an example system for ultrasound imaging, in accordance with an illustrative embodiment;

FIG. 1B is a schematic diagram of the data acquisition and analysis unit of FIG. 1A, in accordance with an illustrative embodiment;

FIG. 1C is a schematic diagram of the ultrasound imaging unit of FIG. 1A, in accordance with an illustrative embodiment;

FIG. 2A is a flowchart of a method for ultrasound imaging, in accordance with an illustrative embodiment;

FIG. 2B is a flowchart of the step of FIG. 2A of performing a calibration procedure, in accordance with an illustrative embodiment;

FIG. 3 is a schematic diagram illustrating steps of the method of FIG. 2, in accordance with an illustrative embodiment;

FIGS. 4A, 4B, and 4C illustrate reconstructed images generated using the method of FIG. 2, in accordance with an illustrative embodiment;

FIG. 5A illustrates reconstructed images generated using the method of FIG. 2, in accordance with another illustrative embodiment;

FIG. 5B illustrates the characterization of the contrast and resolution of the reconstruction of a single scatterer, in accordance with an illustrative embodiment;

FIG. 5C illustrates the contrast-to-noise-ratio (CNR) comparison of compounded ERUI reconstruction, in accordance with an illustrative embodiment; and

FIG. 6 is a block diagram of an example computing device, in accordance with an illustrative embodiment.

It will be noticed that throughout the appended drawings, like features are identified by like reference numerals.

### DETAILED DESCRIPTION

Described herein are systems and methods for ultrasound imaging. In one embodiment, the systems and methods described herein are used for two-dimensional (2D) ultrasound imaging. It should however be understood that the systems and methods described herein may also be used to reconstruct a greater number of pixels, using the same principle, for three-dimensional (3D) ultrasound imaging, which could lead to a greater gain in production cost.

Proposed herein is a technique, referred to as Ergodic Relay Ultrasound Imaging (ERUI), in which images/volumes are obtained using a single receiving element coupled with an ergodic relay. As used herein, the term “image” refers to a 2D digital image while the term “volume” refers to a 3D digital image. In particular, it is proposed herein to insonify at least one object of interest (referred to herein as a “target” or a “sample”) with an emission probe (also referred to herein as an “ultrasonic transmitter”) and to perform signal reception through an ergodic medium (referred to herein as an “ergodic relay”) positioned in front of the reception sensor (also referred to herein as an “ultrasonic receiver”). As used herein, the term “ergodic medium” or “ergodic relay” refers to an acoustic waveguide that provides distinct delay characteristics to any acoustic waves propagating through the waveguide. When considering an ideal ergodic relay, one that is lossless and features perfect reflectors, the acoustic wave at the input point will traverse a specific path to reach a particular output position multiple times, distinct from the paths taken by acoustic waves from other inputs. Leveraging the linear and temporally shift-invariant characteristics of an acoustic ergodic relay, it becomes possible to calibrate its response beforehand. Consequently, the detection of acoustic waves emitted by multiple sources simultaneously can be achieved by employing at least one single-element transducer connected to the ergodic relay. Received signals are encoded using the ergodic relay and images/volumes of the target are decoded by learning a specific and unique signature for each pixel a priori.

The systems and methods described herein rely on a property referred to as the “ergodicity property”, namely the fact that average behavior of a system can be deduced from the trajectory of a typical point. In particular, the ergodicity property ensures that each pixel to be reconstructed will be associated with a unique path (and signature) to the reception channel(s) associated with the ultrasonic receiver. Each signal from each reception channel is the sum of signals from the overall insonified area and, based on the pixels' signature, the associated inverse problem can be solved to separate each pixel's signal. Using the ergodicity property, the proposed ultrasound imaging systems and methods enable the use in reception of few channels, and images/volumes can be reconstructed by several transducer elements (with a collection of similar or different ergodic relays), instead of the thousands of transducer elements used in conventional techniques. In some embodiments, images/volumes can be reconstructed by a single transducer element. As a result, in some embodiments, data transfer rate, cost, and probe complexity can be reduced while increasing the potential use of matrix probes with larger field of views in clinics. In some embodiments, the proposed technique can also achieve image quality comparable to the one obtained with ultrasonic probes having fully populated transducer element arrays.

Furthermore, positioning the ergodic relay in front of the reception sensor, in the manner proposed herein, may reduce the properties, such as the number, intensity, pressure and/or other relevant properties, of the ultrasonic waves that are transmitted to (and interacting with) the object of interest. This may prove beneficial in a variety of applications. For example, in medical applications, reducing the amount of ultrasonic waves that interact with the tissue of a patient may reduce bioeffects, such as rise in temperature and cavitation.

The theory supporting the ergodic ultrasound imaging systems and methods proposed herein will now be described. A forward problem links the measurements after the ergodic relay of all piezoelectric elements y to the medium x via a direct operator describing the emission, receive, and propagation characteristics of an ultrasound sequence as follows:

$$y = G ( x ) \qquad ( 1 )$$

where G is the direct operator, x and y are complex vectors.

Once the problem is set within this formalism, inversion, i.e., finding x knowing y, can be achieved by inverting G. One approach to perform this inversion is to construct a linear approximation to the operator G, denoted K. To construct the operator K, linearity is assumed, where linearity is a good approximation for ultrasound imaging. Under linearity, the Riesz representation theorem applies and allows to represent the operator K by a collection of projections, i.e.

$$∃ ! {k}_{m} ( r ) , {y}_{m} = 〈 {k}_{m} ( r ) \vert x ( r ) 〉 \qquad ( 2 )$$

where yₘ is the m^{th} sample of the acquired data of size M and kₘ is the associated projection. kₘ (r) can be determined, for instance, by setting xₙ=δ(r−rₙ), where r and rₙ represent the position of a scatterer and the position of a given pixel in the image, respectively. This can be done by simulating a single scatterer or by experimentally imaging a small object. In practice, the image space will typically be discretized into N single scatterers (typically located at the center of the desired pixels of the reconstructed image/volume) and all the components k_{m,n} of matrix K can be determined by repeating the operation for each object containing a single non-zero pixel and denoted by xₙ. In matrix notation, one would then obtain:

$$y = {Kx}_{n} = {k}_{n} \qquad ( 3 )$$

where kₙ is the sampled signals of the nth pixels of the calibration image/volume. The entire matrix K can be obtained by repeating the operation for all n. In this case:

$$Y = KI = K \qquad ( 4 )$$

where Y is a matrix containing multiple rows of measurement vectors and I is the identity matrix, i.e. the collection of multiple columns containing canonical vectors xᵢ.

For 2D imaging, the small object can consist in a thin wire in a water bath and positioned perpendicularly to the imaging plane. In 3D, beads smaller than a wavelength embedded in a gel phantom can be used. Other embodiments may apply.

Another approach to construct K consists in using a cloud of strong scatterers (including, but not limited to, microbubbles, metallic particles, or any other suitable strong scatterers) that is simultaneously imaged with an ultrasound probe. As used herein, the term “strong scatterer” refers to an object having dimensions comparable to or smaller than the wavelength of the transmitted ultrasound waves. Also, the term “strong scatterer” refers to an object that reflects ultrasound waves with a high amplitude due to its characteristics, e.g., the impedance, size, resonance and/or other relevant properties. This reference image can be used to determine the exact position of individual microbubbles by localizing the local maxima with subpixel resolution of the image or volume. In this case, each measurement is associated with multiple unique scatterers

$${x}_{l} = {\sum }_{l = 1}^{p} δ ( r - {r}_{l} ) .$$

The difference in position with respect to the center of a pixel can be taken into account using an interpolation term h(x), e.g., by using a phase shift term of

$${e}^{\frac{i 2 π z}{λ}} ,$$

or by forming small pixels that are then used to interpolate to a regular grid. It is desirable for this to be done since, in the context of a cloud of microbubbles, the random position of scatterers cannot be controlled in a way in which they are in the center of the pixels to be reconstructed.

In matrix notation:

$$y = K {\sum }_{l = 1}^{p} {e}_{l} h ( x ) \qquad ( 5 )$$

For multiple measurements:

$$Y = KS \qquad ( 6 )$$

where Y is an M×0 matrix containing the coda for O realization of the cloud of microbubbles and S is an N×0 matrix containing the projection of the actual objects (i.e., the cloud of microbubbles) on the N pixels of the reconstruction grid. One can recover K using the following:

$$\hat{K} = {{YS}^{T} ( {SS}^{T} )}^{- 1} = {{KSS}^{T} ( {SS}^{T} )}^{- 1} = KI \qquad ( 7 )$$

S can either be the reference images of the microbubbles cloud recovered with another ultrasound probe or a projection of the detected microbubbles positions (i.e., using ultrasound localization microscopy (ULM)) on the N pixel grid.

In some embodiments, the reconstruction problem to find y from x, as described in equation (1), can be solved with multiple solvers. Typically, Tikhonov regularization is used, which consists in limiting the L2 norm of the solution

$${min}_{x} ‖ y - Kx ‖ + α ‖ Γ x ‖ ,$$

where ∥.∥ indicates the L2 norm, Γ is a regularization operator and α is a constant. To enable real-time image reconstruction, the following equation, in which the inverse of a matrix is approximated by the inverse of its diagonal, is used:

$$\hat{x} = {( diag ( {K}^{†} K ) )}^{- 1} {K}^{†} y . \qquad ( 8 )$$

It will be appreciated that equation (8) substantially corresponds to the back-projected data divided by the amplitude of a Point Spread Function (PSF) in each pixel.

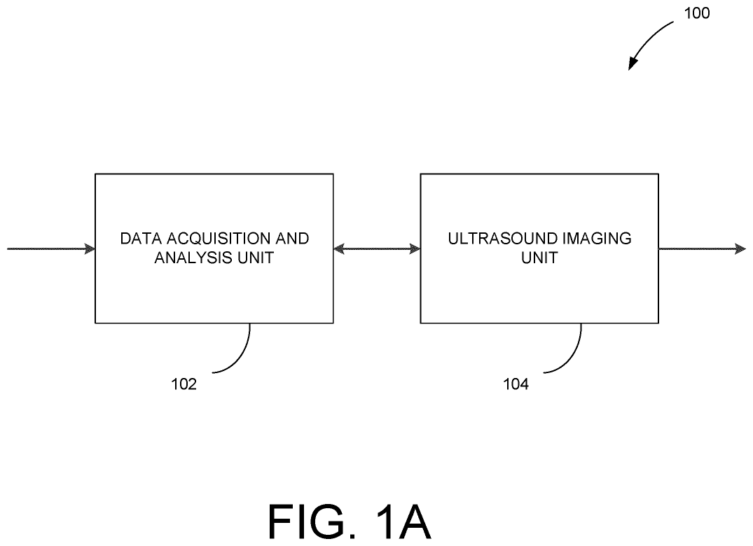

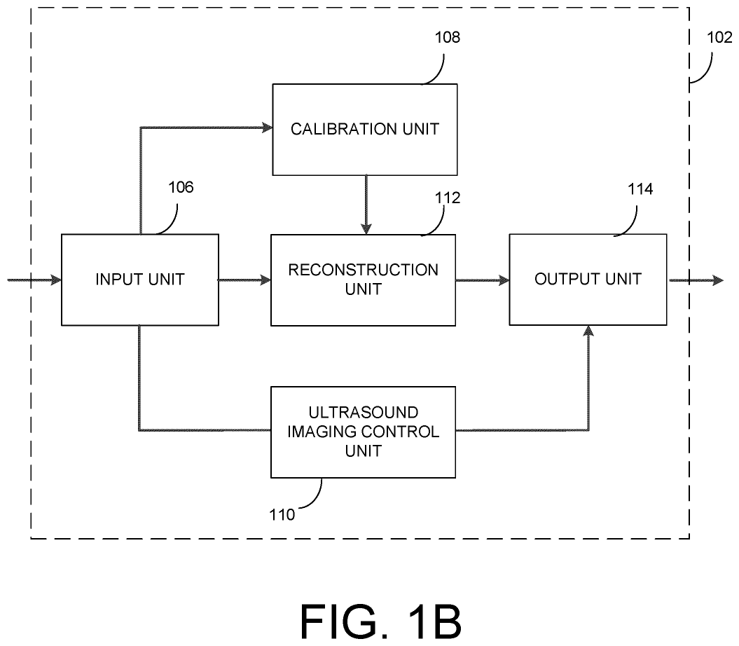

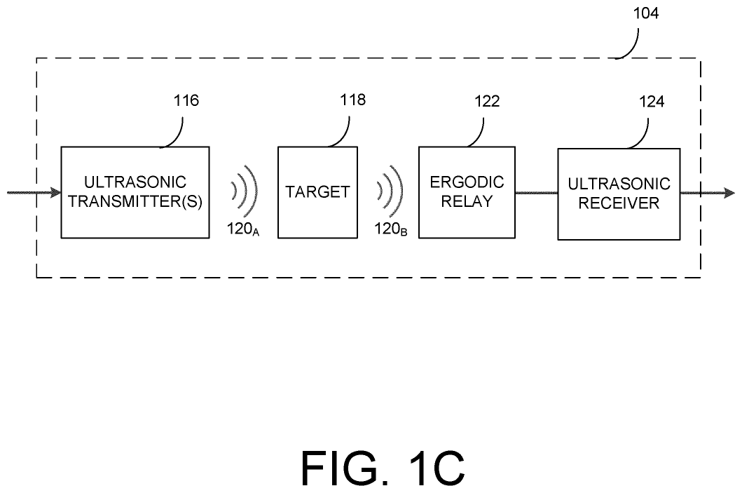

Referring now to FIGS. 1A, 1B, and 1C, a system 100 for ultrasound imaging will now be described, in accordance with one embodiment. The system 100 may be used for performing ERUI using a randomized strong scatterers-based (e.g., microbubbles-based) dictionary. The system 100 illustratively comprises a data acquisition and analysis unit 102 communicatively coupled to an ultrasound imaging unit 104. Any suitable communications means may be used to enable communication between the components of the system 100. For example, wired or wireless communication may be used.

As illustrated in FIG. 1B, the data acquisition and analysis unit 102 comprises an input unit 106, a calibration unit 108, an ultrasound imaging control unit 110, a reconstruction unit 112, and an output unit 114.

The input unit 106 is configured to receive from the ultrasound imaging unit 104 input data (e.g., acoustic signals output by the ultrasound imaging unit 104), as will be discussed further below. The input unit 106 is also configured to receive (via any suitable input device, such as a keyboard, mouse, touchscreen, or the like) input from a user (e.g., an operator) for the purpose of controlling the ultrasound imaging procedure. For example, the input unit 106 may receive from a user a command for controlling operation of the ultrasound imaging unit 104 and such command may be sent to the ultrasound imaging control unit 110 which can accordingly generate control signals for the ultrasound imaging unit 104.

The ultrasound imaging control unit 110 is configured to control operation of the ultrasound imaging unit 104, by outputting control signals thereto. In particular, the ultrasound imaging control unit 110 is configured to send control signals to ultrasonic transmitter(s) (reference 116 in FIG. 1C) of the ultrasound imaging unit 104 to cause the ultrasonic transmitter(s) 116 to transmit at least one focused or unfocused ultrasonic wave towards the target (reference 118 in FIG. 1C). While a single target 118 is illustrated in FIG. 1C, it should be understood that the system 100 may be used for multiple targets simultaneously. The ultrasound imaging control unit 110 is also configured to send control signals to an ultrasonic receiver (reference 124 in FIG. 1C) of the ultrasound imaging unit 104 to cause the ultrasonic receiver 124 to record, in real-time, at least one output signal generated by an ergodic relay (reference 122 in FIG. 1C) encoding a temporal signature to at least one ultrasonic wave backscattered by the target 118 as a function of a location of the target relative to the ergodic relay 122. Although the ergodic relay 122 is illustrated in FIG. 1C as being spaced (i.e. at a distance, not shown) from the target 118, it should be understood that the ergodic relay may, in some embodiments, be in contact with the target 118.

In some embodiments, the calibration unit 108 is configured to perform a calibration procedure (prior to the image/volume reconstruction phase being performed by the reconstruction unit 112) in order to create a dictionary of reception signals (also referred to herein as “calibration codas” or “codas”) that are used to reconstruct images/volumes of a target being imaged. For this purpose, in one embodiment, the calibration unit 108 uses a randomized approach to calibration in which the calibration unit 108 acquires codas associated with different positions of a random point source. In one embodiment, the calibration unit 108 is configured to perform the calibration procedure using a cloud of strong scatterers, for example microbubbles, that are simultaneously imaged. The calibration procedure may be performed using the ultrasound imaging unit 104, where the target 118 is replaced by a medium containing a cloud of microbubbles, and the ultrasonic transmitter(s) 116 and the ultrasonic receiver 124 are replaced by one or more ultrasonic probes (e.g., linear probes). Once the calibration procedure is completed, the object being imaged is used as the target 118 and the linear probes may be replaced. The calibration codas may then be sent from the calibration unit 108 to the reconstruction unit 112. In other embodiments, the calibration codas may be stored in memory (or any other suitable storage means) for subsequent retrieval by the reconstruction unit 112.

Microbubbles are clinically approved contrast agents used routinely in ultrasound imaging to improve the detection of vasculature. The ease with which microbubbles are detected using ultrasound imaging comes from multiple phenomena including the impedance mismatch between the microbubbles and the surrounding environment, which generate strong backscattering of the incident imaging wave, as well as their nonlinearity. In one embodiment, to image such microbubbles, an ultrasonic probe was submerged in a degassed, 12-liter water tank containing 1.08×10⁷ Definity microbubbles (Lantheus Medical Imaging, USA) and was aiming at an ultrasound absorber laying at the bottom of the water tank. In this embodiment, reconstruction was performed during the calibration phase (i.e. by the calibration unit 108) using both a delay-and-sum algorithm (DAS) algorithm and ERUI.

By locating the centroids of sparse scatterers circulating in the vascular network, ULM allows to go beyond the limits of conventional ultrasound imaging fixed by diffraction, and to go down to a resolution of only a few microns, using microbubbles, or sono-activated nanodroplets. In addition to its high imaging rate, low cost, non-invasiveness and non-ionization, this modality is capable of imaging the entire vasculature of an organ within a wide field of view and in depth.

To localize the microbubbles, the calibration unit 108 may use any suitable pipeline. In one embodiment, the PSF of a microbubble located in the center of the reconstructed region was simulated considering a fully populated array and correlated with the reconstructed images/volumes. A Gaussian fitting was then performed on the correlation maps to localize microbubbles centers with a subwavelength precision. The microbubbles were tracked in time using a nearest-neighbor criterion to eliminate microbubbles that did not persist for more than two consecutive frames.

In some embodiments, the calibration unit 108 may apply harmonic imaging techniques during the calibration procedure. Harmonic imaging techniques take advantage of the nonlinear oscillations exhibited by microbubbles or tissue when sufficient pressure is achieved. In one embodiment, a 2.5 MHz monoelement (Panametrics V306-0.5″) was used to emit a pulsed plane wave and a 5 MHz linear probe (L7-4, ATL Philips, WA) on which an ergodic relay (as in 122) was affixed and used as a receiver. The matrix K was generated using the cloud of microbubbles described herein above. The collection of resonant signals from the microbubbles Y was acquired using the 5 MHz probe while the position associated with microbubbles S was determined with a second 2.25 MHz probe (C4-2, ATL Philips, WA) placed in the same imaging plane as the emission probe. The position of each microbubble was computed by the calibration unit 108 using the methods described herein above. In the image/volume reconstruction unit, only the 2.5 MHz monoelement and 5 MHz linear probe (L7-4, ATL Philips, WA) on which an ergodic relay (as in 122) was affixed and used as an emitter and receiver, respectively.

In one embodiment of a Harmonic imaging technique, the ultrasonic transmitter(s) 116 are configured to transmit an excitation wave with an amplitude that is sufficient for harmonics of a central emission frequency to be generated. In this case, the ultrasonic receiver 124 is configured to receive echoes returned from the target 118 at a receive frequency bandwidth that contains one or more integer multiples of the central emission frequency. It will be appreciated that the amplitude of the excitation waves transmitted to a strong scatterer is generally lower than the amplitude of the excitation waves transmitted to conventional samples (e.g. body tissues).

In some embodiments, for 3D imaging, calibration may also be performed by the calibration unit 108 using pseudo-random distribution of ultrasound contrast agents, allowing strong ultrasound signals acquisition. The calibration unit 108 may be developed as a platform configured for the calibration to freely move the probe according to specific reception strategies that use averaging to achieve high signal to noise ratios.

In some embodiments, the calibration unit 108 may use deep learning algorithms to perform the calibration procedure. The inversion of the matrix of the encoder of positions of microbubbles S, used to find a system matrix K as described in equation (7) and the decoder of the inverse problem of (1) assumes linearity between the vectors. Hence, it is also possible to replace the linear encoder-decoder with a non-linear encoder-decoder based on deep learning. In one embodiment, a convolutional neural network (CNN) with a U-Net architecture could be used. In such an embodiment, during the calibration phase, ultrasound signals acquired via the ergodic relay as in 122 from multiple microbubbles flowing freely through water were used as the input data of an encoder network of the calibration unit 108, the encoder network capturing frames and temporal information into latent feature layers. An expanding decoder network was then used to detect positions of the microbubbles.

It should be understood that, in addition to being used for performing calibration, any suitable machine learning technique(s) (e.g., neural networks) may be used to perform the imaging procedure (i.e. reconstructing an image of a target) described herein.

In one embodiment, the calibration procedure may be performed by the calibration unit 108 using a dilated convolution kernels architecture. In such a dilated CNN, the dilation rate (i.e. the spacing between the kernel elements) increases with increasing network depth d, where d=2m, with me being the layer index, starting from m=0. Provided a stride of 1 is used in all convolutions, such a dilation rate prevents loss of resolution or coverage. In the dilated CNN, the first convolutional layer connects a single input channel to 64 channels. All intermediate convolutional layers connect 64 channels to 64 channels. Each convolutional layer is followed by a batch normalization layer (to speed up training) and a Rectified Linear Unit (ReLU) activation layer. The final layer is a fully connected layer to output each pixel of the reconstructed image. The dilated CNN may be trained in any suitable manner. For example, the CNN may be trained on 10900 of 15625 encoded signals and their respective positions from the dictionary of reception signals, and each model may be trained for 500 epochs using a batch size of 16. The dilated CNN and the training may then be implemented in Python using PyTorch, and the Adam optimization algorithm with a learning rate of 0.01 may be used to optimized the parameters of the CNN. A L2 loss may be applied on each pixel in regards to the true position of the single scatterer in calibration.

It should be understood that the description above is for illustrative purposes only and other embodiments may apply. Indeed, since the ergodicity property is independent of the transmitted signal, it is possible to take advantage of other types of transmissions, for example, broadband signals frequencies, random, chirps etc. Chirps allow to have a wider frequency content, reduce the lobes, increase the signal to noise ratio (SNR) and increase voltage virtually without changing the mechanical stress applied to the microbubbles, which further increases the SNR of the signatures and improves the resolution and localization of the microbubbles used during calibration. Other techniques such as Hadamard encoding of the emission pulse can also be leveraged. It should also be understood that any suitable strong scatterers (e.g., metallic particles, or the like) other than microbubbles may apply.

The reconstruction unit 112 is configured to perform operations to reconstruct images/volumes using the calibration codas (e.g., received from the calibration unit 108 or retrieved from storage) and the data in the real time acoustic signals received from the ultrasound imaging unit 104. For this purpose, the reconstruction unit 112 uses the following equation:

$$s ( \vec{r} ) = {\sum }_{t} {e}_{\vec{r}} ( t ) \times s ( t ) \qquad ( 8 )$$

where s(\vec{r}) is the reconstructed image/volume of the target 118, s(t) is the signal received from the ultrasound imaging unit 104 and e_{\vec{r}}(t) is the dictionary of reception signals (or calibration codas) determined by the calibration unit 108 from a unique point reflector located at. In some embodiments, the signals e_{\vec{r}}(t) are encoded on a number of bits lying in the range 1 to 64. In one embodiment, the signals e_{\vec{r}}(t) are encoded on 1 bit.

The output unit 114 is configured to receive control signals from the ultrasound imaging control unit 110 and to transmit, using any suitable communication means, the control signals to the ultrasound imaging unit 104 to control operation thereof. The output unit 114 is also configured to receive the reconstructed images/volumes from the reconstruction unit 112 and to cause the images/volumes to be rendered via any suitable output means, e.g., a display, or the like. In some embodiments, the output unit 114 may also be configured to obtain from the reconstruction unit 112 raw data from the acoustic signals and to output the raw data using any suitable output means.

Referring now to FIG. 1C, during the ultrasound imaging procedure in which an object of interest (i.e. the target 118) is being imaged, the ultrasound imaging unit 104 is configured to use a plane wave to insonify the entire field of view (FOV) at a high frame rate (e.g., a frame rate above 1000 Hz). The target 118 may comprise any suitable target to be imaged including, but not limited to, a bodily sample.

The ultrasound imaging unit 104 comprises ultrasonic transmitter(s) 116 for transmitting towards the target 118 being imaged at least one first ultrasonic wave 120_{A}. In one embodiment, the at least one first ultrasonic wave 120_{A} is a plane wave. The at least one first ultrasonic wave 120_{A} may be focused or unfocused. The ultrasound imaging unit 104 also comprises an ergodic relay 122 coupled to the target 118 and configured to receive at least one second ultrasonic wave 120_{B} backscattered by the target 118 and encode (i.e. apply) a temporal signature to the at least one backscattered wave 120_{B} as a function of a location of the target 118 relative to the ergodic relay 122 for generating at least one output signal. As can be seen in FIG. 1C, the target 118 is positioned between the ultrasonic transmitter(s) 116 and the ergodic relay 122, along the paths of ultrasonic waves 120_{A} and 120_{B}. The ultrasound imaging unit 104 further comprises an ultrasonic receiver 124 coupled to the ergodic relay 122 and configured to receive the at least one output signal therefrom. Although FIG. 1C illustrates the ultrasonic transmitter(s) 116 as being separate (i.e. disjointed) from the ultrasonic receiver 124, it should be understood that, in other embodiments, the ultrasonic transmitter(s) 116 and the ultrasonic receiver 124 may be provided in a same device.

The ultrasonic transmitter(s) 116 may be positioned at any suitable location relative to the target 118. In some embodiments, the ultrasonic transmitter(s) 116 may be distanced axially from the target 118. The ultrasonic transmitter(s) 116 are configured to generate at least one ultrasonic wave as in 120_{A} towards the target 118. The transmitted ultrasonic wave has a given transmission frequency in the target medium which results in at least one ultrasonic wave as in 120_{B} being backscattered by the target 118 in directions away from the insonified area of the target 118, including in a direction towards the ergodic relay 122.

In one embodiment, the ultrasonic transmitter(s) 116 comprise at least one array of ultrasonic transducer elements (e.g., one or more linear transducer arrays, one or more two-dimensional transducer arrays, or any combination thereof). It should be understood that, in other embodiments, the ultrasonic transmitter(s) 116 may comprise a single ultrasonic transducer element. In particular, in some embodiments, the ultrasonic transmitter(s) 116 may comprise a single mono-element used to transmit a plane or diverging wave front as in 120_{A} into the target 118 and the ultrasonic wave 120_{B} backscattered by the target 118 may be received using the ergodic relay 122. Other embodiments may apply. For example, in other embodiments, rather than using the ultrasonic transmitter(s) 116, the ergodic relay 122 may be used with the appropriate calibration coda to transmit a plane or diverging wave front as in 120_{A} into the target 118 and the ultrasonic wave 120_{B} backscattered by the target 118 may also be received using the ergodic relay 122. More specifically, the ergodic relay 122 may be configured to project, from one or more output locations of the ergodic relay 122, one or more ultrasonic waves towards the target 118, and to receive one or more backscattered ultrasonic waves at one or more input locations of the ergodic relay 122 different from the one or more output locations. In both embodiments, an image/volume of the target 118 is then reconstructed with the appropriate dictionary, using the reconstruction unit 112 in the manner described herein above.

In one embodiment, the ultrasonic receiver 124 comprises a single transducer element coupled to the ergodic relay 122 at one or more output locations thereof. The ultrasonic receiver 124 may be coupled directly to (i.e. touching) a surface of the ergodic relay 122 or coupled thereto via any suitable coupling material, such as a resin, a gel, silicon water, or the like, in a manner that enables the ultrasonic receiver 124 to detect encoded signals output by the ergodic relay 122. In other embodiments, the ergodic relay 122 is designed to be apart from the ultrasonic receiver 124 and coupled thereto (indirectly) using any suitable means. In yet other embodiments, the ergodic relay 122 may be integrated with the ultrasonic receiver 124 and the ultrasonic transmitter(s) 116 as a single device. It should also be understood that, although the ergodic relay 122 is illustrated in FIG. 1C as being coupled to a single ultrasonic receiver 124, it should be understood that the ergodic relay 122 may be coupled to multiple ultrasonic receivers as in 124.

Any suitable device or combination of devices may be used to implement the ergodic relay 122. In addition, the ergodic relay 122 may have any suitable shape and size, and may comprise any suitable material. For example, the ergodic relay 122 may comprise, but is not limited to, a prism (e.g., a right-angle prism made of fused silica), a plate (e.g., a glass plate, a quartz plate, a slide, a coverslip, etc.), or silicon water. In some embodiments, the ergodic relay 122 may have a different (e.g., greater) size than the ultrasonic receiver 124. In some embodiments, the ergodic relay 122 comprises a so-called “rod forest”, which may be used to used to minimize impedance cutoff, thereby improving the system's overall efficiency. The rod forest illustratively comprises a cavity filled with a fluid combined with a high-order multiple scattering medium. In some embodiments, the rod forest consists of a water filled metallic cavity comprising a plurality of metallic rods arranged perpendicular to the direction of propagation of the ultrasound waves. The rods may be separated from one another by any suitable distance, may have any suitable dimension (e.g., radius, length), and may be made of any suitable material. In other embodiments, the ergodic relay 122 has a shape of a rectangular parallelepiped having a recess formed therein, the recess having the shape of a portion of a sphere. Other embodiments may apply.

The ergodic relay 122 is a low-loss propagation medium that is used as an encoder to transform the backscattered ultrasonic wave 120_{B} into unique temporal signals. In particular, the internal boundaries (i.e. inner surfaces) of the ergodic relay 122 are used to reflect the backscattered ultrasonic wave 120_{B}, which scrambles (i.e. encodes a characteristic temporal signature to) the ultrasonic wave 120_{B} based on its input location relative to the ergodic relay 122 and generates encoded output signals. The backscattered ultrasonic wave 120_{B} is reflected at the boundaries of the ergodic relay 122 due to the discontinuity in transmissivity between the surrounding air (or other medium) outside the boundaries and the medium of the ergodic relay 122. In one embodiment, the ergodic relay 122 is assumed to be lossless and its boundaries are considered to be perfect reflectors, such that an ultrasonic wave as in 120_{B} at a particular input location of the ergodic relay 122 propagates to an output location of the ergodic relay 122 along a unique path relative to the paths of other ultrasonic waves at other input locations of the ergodic relay 122. The ergodic relay 122 thus outputs encoded signals after multiple time delays relative to the delivery of the ultrasonic wave 120_{B} at input location(s) to the ergodic relay 122. In other words, the ergodic relay 122 encodes the spatial location of the backscattered ultrasonic wave 120_{B} as a characteristic temporal signature which is formed between the time of delivery of the ultrasonic wave 120_{B} and the time at which the output signal is received by the ultrasonic receiver 124 from the ergodic relay 122.

In one embodiment, the ergodic relay 122 is linear and temporally shift-invariant, such that the temporal signature of the ergodic relay 122 for the ultrasonic wave paths can be calibrated by the calibration unit 108. The response of each input position of the ergodic relay 122 can indeed be recorded in advance during a calibration process (e.g., performed at the calibration unit 108, as described herein above) and the temporal signature of the ergodic relay 122 can be established. Using the calibrated responses (i.e. the dictionary of reception signals) determined by the calibration unit 108, the reconstruction unit 112 is configured to analyze the encoded signals (i.e. ultrasonic signals encoded with a temporal signature due to each signal's internal path within the ergodic relay 122) output by the ergodic relay 122 (and received at the ultrasonic receiver 124) to determine their input locations. The reconstruction unit 112 is configured to mathematically decode the encoded signals to reconstruct an image/volume of the target 118, using equation (8) above. In particular, since each signal's internal path is associated with a particular location of the target 118, the reconstruction unit 112 is able to map each encoded signal to the location of the target 118 based on the signal's temporal signature.

It will be understood that the system 100 for ultrasound imaging presented in FIG. 1C is not limited to comprising a single ergodic relay 122 and a single ultrasonic receiver 124. Indeed, in some embodiments, the system 100 may comprise a plurality or ergodic relays and a plurality of ultrasonic receivers. In this case, each ergodic relay can be coupled to one or more ultrasonic receivers and each ultrasonic receiver can be coupled to one or more ergodic relays. The type of ergodic relay and/or ultrasonic receiver may vary for each coupling.

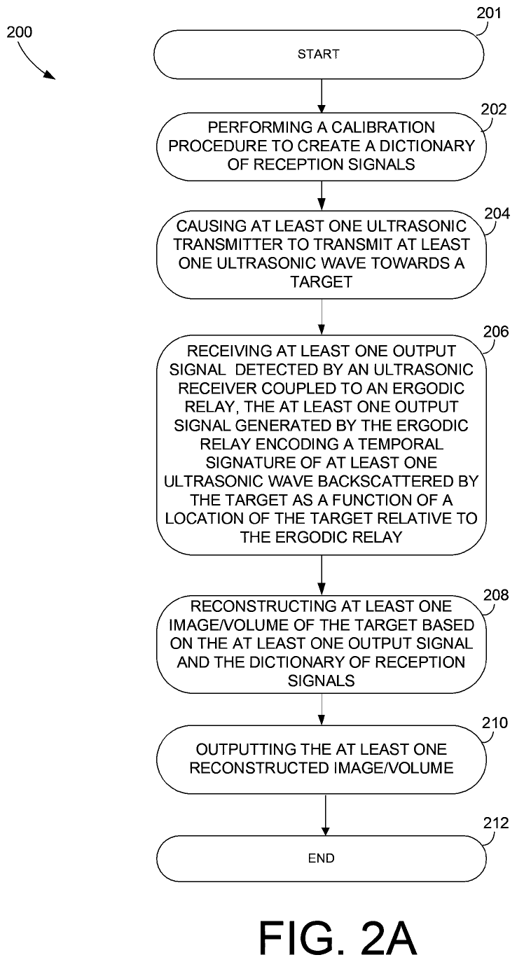

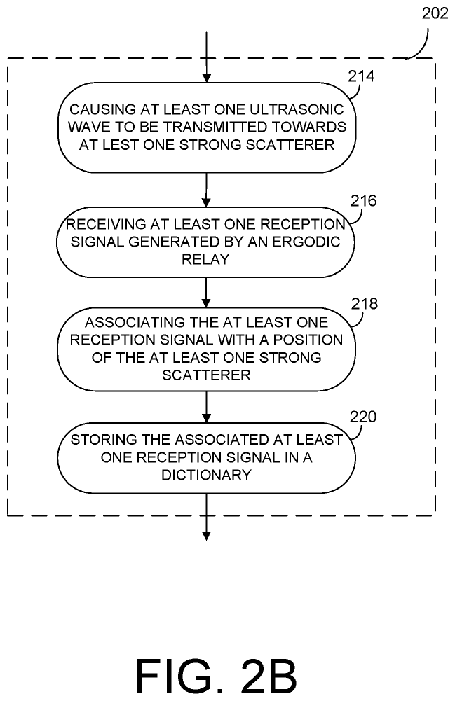

Referring now to FIG. 2A, a method 200 for ultrasound imaging will now be described, in accordance with one embodiment. The method 200 starts at step 201. At step 202, a calibration procedure is performed (e.g., by the calibration unit 108 of FIG. 1B) to create a dictionary of reception signals in the manner described herein above. At step 204, at least one ultrasonic transmitter (e.g., the ultrasonic transmitter(s) 116 of FIG. 1C) is caused (e.g., by the ultrasound imaging control unit 110 of FIG. 1B) to transmit at least one ultrasonic wave towards a target (e.g., the target 118 of FIG. 1C). At step 206, at least one output signal detected by an ultrasonic receiver (e.g., the ultrasonic receiver 124 of FIG. 1C) coupled to an ergodic relay (e.g., the ergodic relay 122 of FIG. 1C) is received (e.g., at the reconstruction unit 112 of FIG. 1B). In one embodiment, the at least one output signal is received at step 206 in real-time and recorded for all pixels. As described herein above, the at least one output signal is generated by the ergodic relay encoding a temporal signature to at least one ultrasonic wave backscattered by the target as a function of a location of the target relative to the ergodic relay. At step 208, at least one image/volume of the target is reconstructed (e.g., at the reconstruction unit 112) based on the at least one output signal and the dictionary of reception signals. This may be achieved using equation (8) above, by correlating the at least one output signal with the reception signals stored in the dictionary. The at least one reconstructed image/volume can then be output at step 210, using any suitable output means, such as a display associated with a computing device configured to perform the method 200 and/or implement the system 100. At step 212, the method 200 ends.

Referring now to FIG. 2B, the step 202 of performing a calibration procedure comprises, at step 214, causing at least one ultrasonic wave to be transmitted towards at least one strong scatterer. The at least one strong scatterer may be, for instance, a plurality of microbubbles in a water tank, as described herein above. In some embodiments, the at least one ultrasonic waves are directed towards a volume comprising the at least one strong scatterer. In this case, the spatial coordinates of the at least one strong scatterer are known, such that the position of the at least one strong scatterer is predetermined and can be associated with at least one reception signal.

At step 216, at least one reception signal generated by an ergodic relay coupled to the at least one strong scatterer is received. In one embodiment, the at least one reception signal is received at step 216 in real-time and recorded for each pixel individually. The ergodic relay may be as described herein above. At step 218, the at least one reception signal is associated with a position of the at least one strong scatterer. At step 220, the associated at least one reception signal is stored in a dictionary. In one embodiment, the dictionary is stored in a memory (or any other suitable storage means) for subsequent retrieval. It should be understood that steps 214 to 220 may be performed as many times as required in order to build the dictionary of reception signals.

It will be appreciated that the criteria for a dictionary to be created during the calibration procedure performed at step 202 may vary. In one embodiment, the dictionary is created once the acoustic signature of each pixel of an image/volume is known. In other embodiments, the dictionary is created once signal to noise ratios of arbitrary pixels in an image/volume reach a given (e.g., predefined) threshold. It will be further appreciated that a calibration method such as the method 200 can be used to calibrate an ultrasound imaging system for multiple uses on various objects, without having to recalibrate the system before each measurement. In other words, the calibration procedure performed at step 202 is not specific to a given object and may be performed once rather than multiple times.

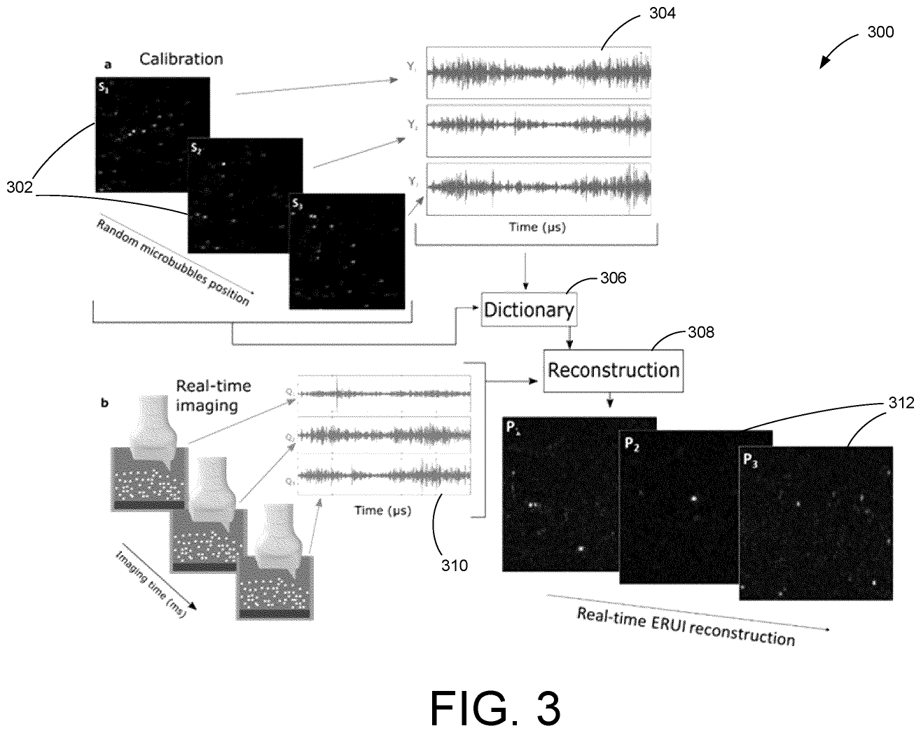

FIG. 3 is a schematic diagram 300 illustrating the method 200 of FIG. 2A. As can be seen from FIG. 3, in one embodiment, the calibration procedure performed at step 202 comprises using reference images 302 and associated signals 304 generated by imaging clouds of strong scatterers, for example microbubbles, with an ultrasound probe to create a dictionary 306. During reconstruction 308, the dictionary 306 and the signals 310 output by the ergodic relay (and detected by the ultrasonic receiver) are used to generate reconstructed images 312.

To demonstrate the capability of the systems and methods proposed herein, a right-angle prism made of ultraviolet (UV) fused silica (PS615, Thorlabs, Inc.) was affixed to a 5-MHz linear probe (L7-4, ATL Philips, WA) to be used as the ergodic relay 122. 64 of the 128 elements of the probe were used to emit tilted plane waves, and signals were then recorded on all the elements of the probe, including the one affixed to the prism. To form images/volumes using such an ergodic relay 122, the direct matrix K was first built by experimentally acquiring codas (i.e., signals measured by the ergodic relay 122) associated with point-sources (e.g. a 20-um wire in a water tank) in each pixel to be reconstructed. Images/volumes could then be obtained by applying the transpose operation to the direct matrix. In this embodiment, images/volumes were reconstructed using both a DAS algorithm and ERUI. It should however be understood that, in some embodiments, only ERUI may be used. The DAS algorithm ran on a graphical processing unit from a Matlab interface (R2022a, Mathworks, MA). The method was implemented to reconstruct two 20 um diameter wires, spaced 1.5 mm apart axially and to image microbubbles diluted in a water tank.

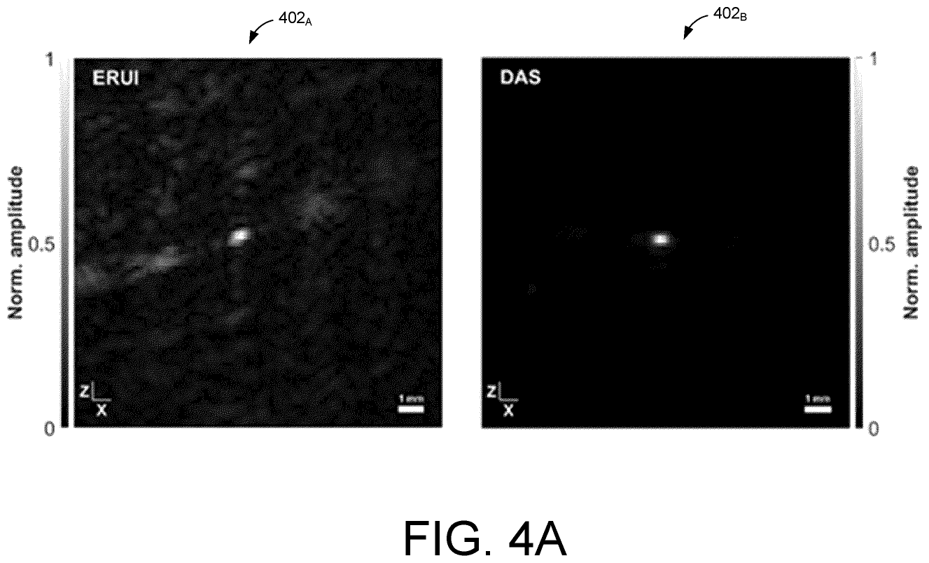

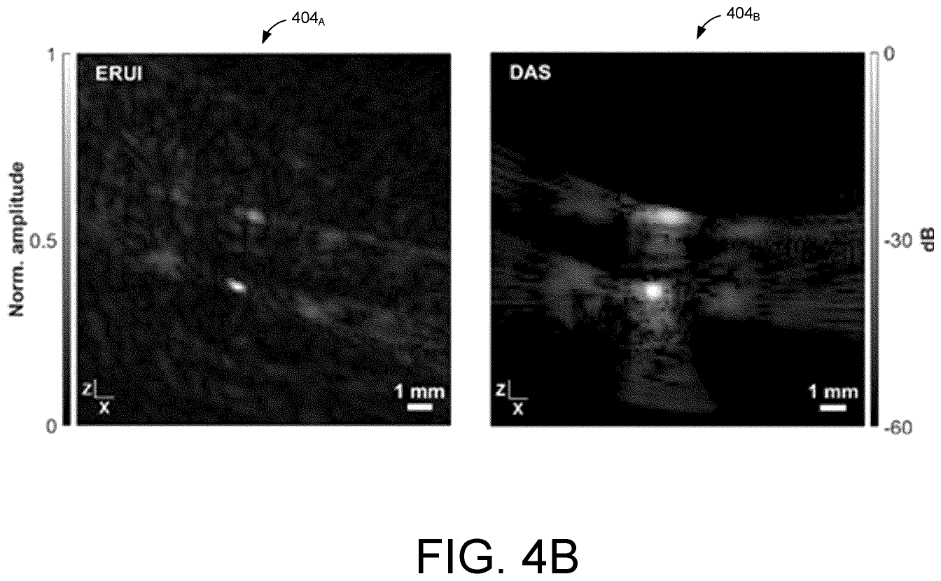

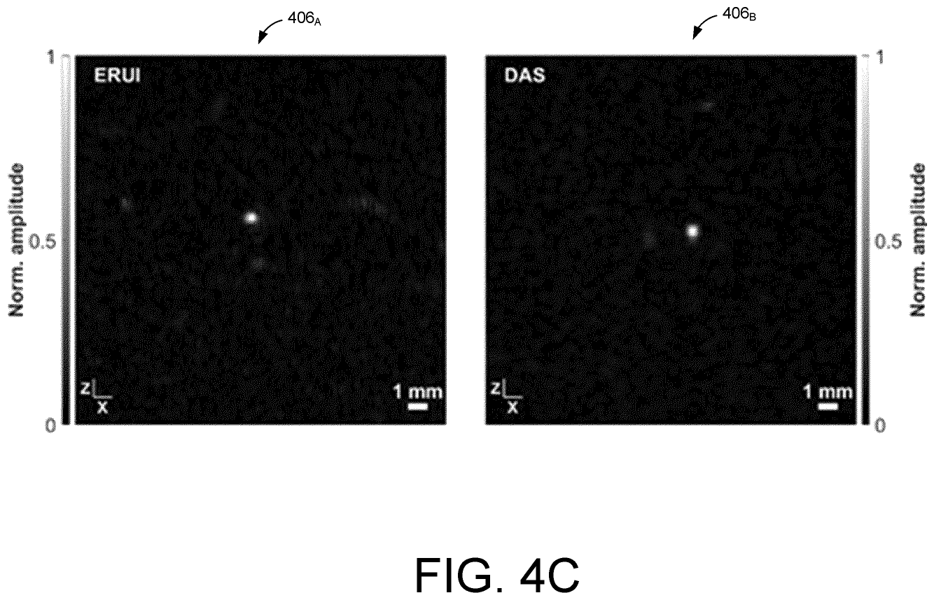

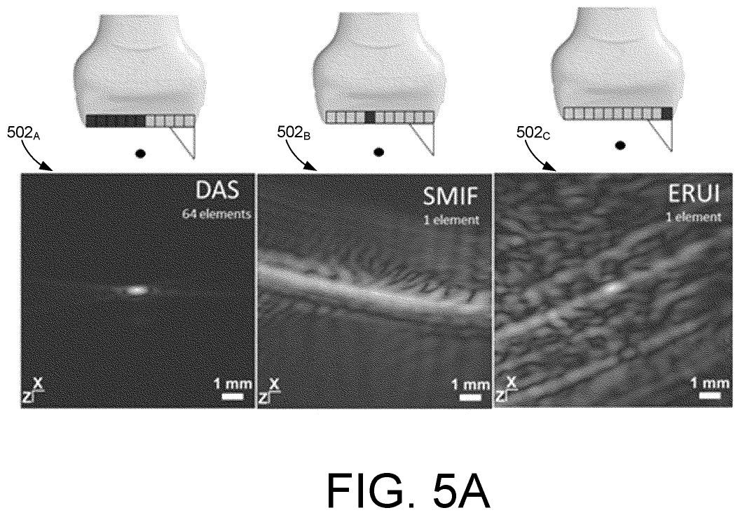

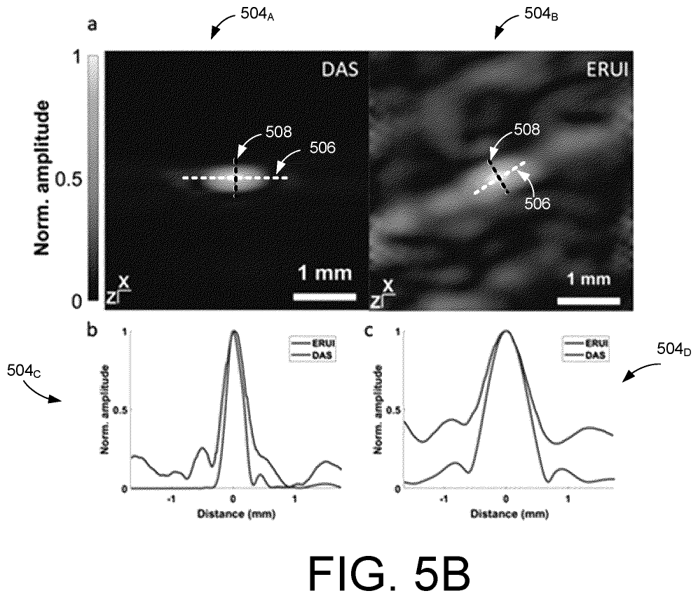

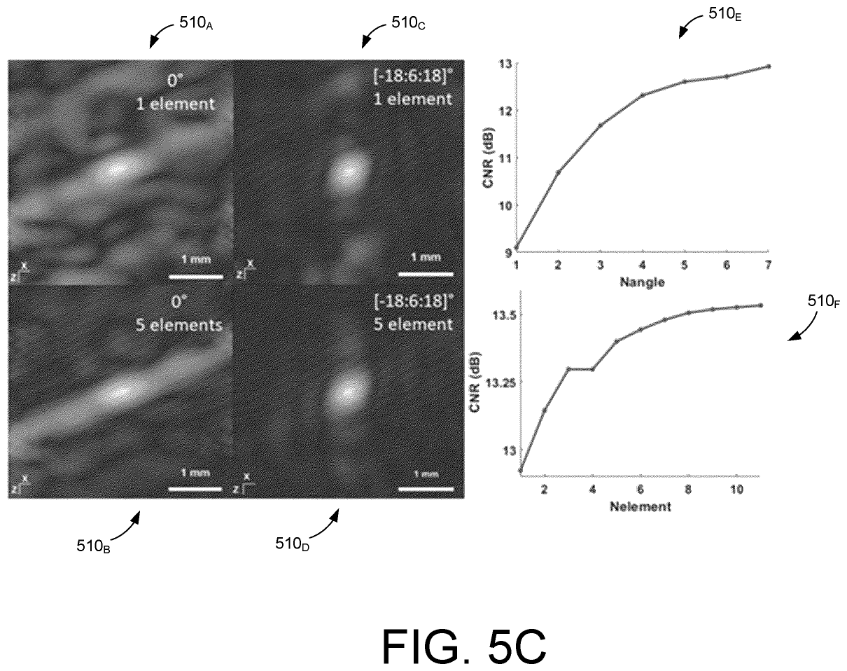

Referring now to FIGS. 4A-4C and FIGS. 5A-5C, results obtained using the systems and methods proposed herein will now be described. FIG. 4A illustrates an image 402_{A} of a single microbubble in a water tank reconstructed using ERUI, and an image 402_{B} of a single microbubble in a water tank reconstructed using DAS. FIG. 4B illustrates an image 404_{A} of two 20 um diameter wires, spaced 1.5 mm apart axially reconstructed using ERUI, and an image 404_{B} of two 20 um diameter wires, spaced 1.5 mm apart axially reconstructed using DAS. FIG. 4C illustrates an image 406_{A} of a single microbubble in a water tank reconstructed using ERUI with a microbubbles-based learning method, and an image 406_{B} of a single microbubble in a water tank reconstructed using DAS.

FIG. 5A illustrates the reconstruction of a single thin wire using various reconstruction methods. The image 502_{A} is obtained with a DAS algorithm using the same elements used for transmitting the emission pulse. The image 502_{B} is obtained using a spatiotemporal matrix image formation (SMIF), where the matrix K is defined by the measurement of a single element not covered by the ergodic relay. The image 502_{C} is obtained with ERUI reconstruction using a single element covered by the ergodic relay. From FIG. 5A, it can be seen that DAS with 64 elements and ERUI with a single element can clearly reconstruct the scatterer, while the SMIF reconstruction cannot localize the scatterer.

FIG. 5B illustrates the contrast and resolution of the reconstruction of a single scatterer. The image 504_{A} shows DAS reconstruction with normalized amplitude of a single scatterer. The image 504_{B} shows ERUI reconstruction. For DAS and ERUI, the resolution and contrast measurement were performed with lateral resolution of the scatterer, taken to be parallel to the angle of the prism for the ergodic relay image. In images 504_{A} and 504_{B}, dotted lines 506 represent the lateral direction taken for contrast and resolution measurements, while dotted lines 508 indicate the axial direction. The graph 504_{C} of FIG. 5B is an axial profile taken along the lines 508 of images 504_{A}, 504_{B}, while the graph 504_{D} in FIG. 5B is a lateral profile taken along the lines 506 of images 504_{A}, 504_{B}. From FIG. 5B, one can see that the lateral resolution of the scatterer with ERUI is about 1 wavelength and is close to the one obtained with DAS. In the axial direction, a single element ergodic relay image also has a resolution comparable to the DAS image. Moreover, it can be seen that, for both the lateral and axial directions, contrast with ERUI is higher than the one observed with DAS.

To compare the increase in performance gained by ERUI with multiple ergodic signals, the single scatterer was imaged using multiple transmission angles and multiple received spatiotemporal encoding codas associated with multiple receiving elements. Contrast gained was calculated for the compounding of images obtained with each angle and encoding element. FIG. 5C illustrates the contrast-to-noise-ratio (CNR) comparison of compounded ERUI reconstruction. Images 510_{A}, 510_{B} are obtained using a probe having one element and a probe having five elements, respectively, at an angle of transmission of zero degrees. The images 510_{C}, 510_{D} are obtained using a probe having one element and a probe having five elements, respectively, at angles of transmission between minus eighteen degrees to eighteen degrees with increments of six degrees. The graph 510 shows the CNR as a function of the angle of transmission, while the graph 510 shows the CNR as a function of the number of elements.

As can be seen from FIGS. 4A-4C and FIGS. 5A-5C, ERUI enabled the reconstruction of images/volumes that were comparable with DAS with ultrasound probes using arrays having 64 elements. Resolutions achieved were in accordance with the theoretical limit for the aperture size the prism. While the prototype was based on a conventional probe, low-cost alternatives that sample only one channel rather than the entire probe could be considered. These results show that, by reducing to a single element a probe usually made up of between a hundred and a thousand ultrasonic elements for 2D and respectively, it is possible to obtain significant gains in the complexity of the electrical connectivity, in the material cost as well as the amount of data to be acquired. In one embodiment, the systems and methods proposed herein may be used to solve the current limitations of ultrasound imaging, create new potential clinical applications, and allow a wider use of ultrasound imaging in biomedical, consumer electronic, and industrial fields (e.g., non-destructive testing), amongst others. It should also be understood that other uses including, but not limited to, localization microscopy, may apply.

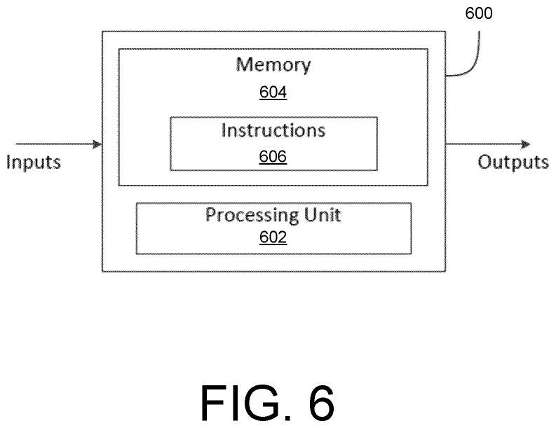

FIG. 6 is a schematic diagram of computing device 600, which may be used to implement at least part of the system 100 (e.g., the data acquisition and analysis unit 102) and/or method 200 of FIG. 2A. The computing device 600 comprises a processing unit 602 and a memory 604 which has stored therein computer-executable instructions 606. The processing unit may 602 may comprise any suitable devices configured to implement the functionality of the method 200 such that instructions 606, when executed by the computing device 600 or other programmable apparatus, may cause the functions/acts/steps performed by method 200 as described herein to be executed. The processing unit 602 may comprise, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 604 may comprise any suitable known or other machine-readable storage medium. The memory 604 may comprise non-transitory computer readable storage medium, for example, but not limited to, an electronic, magnetic, optical, electromagnetic, infrared, or semiconductor system, apparatus, or device, or any suitable combination of the foregoing. The memory 604 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically erasable programmable read-only memory (EEPROM), Ferroelectric RAM (FRAM) or the like. Memory 604 may comprise any storage means (e.g., devices) suitable for retrievably storing machine-readable instructions 606 executable by the processing unit 602.

The above description is meant to be exemplary only, and one skilled in the art will recognize that changes may be made to the embodiments described without departing from the scope of the invention disclosed. Still other modifications which fall within the scope of the present invention will be apparent to those skilled in the art, considering a review of this disclosure.

Various aspects of the systems and methods described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although embodiments have been shown and described, it will be apparent to those skilled in the art that changes, and modifications may be made without departing from this invention in its broader aspects. The scope of the following claims should not be limited by the embodiments set forth in the examples but should be given the broadest reasonable interpretation consistent with the description.

1. A system for ultrasound imaging, the system comprising:
at least one ultrasonic transmitter configured to transmit at least one ultrasonic wave towards at least one target;
an ergodic relay coupled to the at least one target, the ergodic relay configured for receiving at least one ultrasonic wave backscattered by the at least one target and for applying a temporal signature, as a function of a location of the at least one target relative to the ergodic relay, to the at least one backscattered wave to generate at least one output signal; and
an ultrasonic receiver coupled to the ergodic relay and configured to receive the at least one output signal therefrom, the at least one output signal used to reconstruct at least one image of the at least one target.

2. The system of claim 1, wherein the at least one ultrasonic transmitter is configured to transmit the at least one ultrasonic wave having a given transmission frequency in the at least one target.

3. The system of claim 1, wherein the at least one ultrasonic transmitter comprises an array of ultrasonic transducer elements.

4. The system of claim 1, wherein the ultrasonic receiver comprises a single ultrasonic transducer element.

5. The system of claim 1, wherein the ergodic relay comprises a right-angle prism.

6. The system of claim 1, further comprising a computing device communicatively coupled to the ultrasonic receiver, the computing device configured to receive the at least one output signal from the ultrasonic receiver and to reconstruct the at least one image of the at least one target based on the at least one output signal and a dictionary of reception signals.

7. The system of claim 6, wherein the computing device is configured to reconstruct the at least one image of the at least one target as follows:
$$s ( \vec{r} ) = {\sum }_{t} {e}_{\vec{r}} ( t ) \times s ( t )$$
where s(\vec{r}) is the at least one image of the at least one target as reconstructed, s(t) is the at least one output signal, and e_{\vec{r}}(t) is the dictionary of reception signals determined from a unique point reflector located at f.

8. The system of claim 6, wherein the computing device is configured to determine the dictionary of reception signals during a calibration procedure in which at least one strong scatterer is imaged with an ultrasound probe to generate the reception signals and at least one reference image associated therewith.

9. The system of claim 6, wherein the computing device is configured to apply at least one deep learning technique to at least one of reconstruct the at least one image of the at least one target and perform a calibration procedure to determine the dictionary of reception signals.

10. The system of claim 8, wherein the computing device is configured to use a dilated convolution kernels architecture for performing the calibration procedure.

11. The system of claim 1, wherein the ergodic relay is configured for applying the temporal signature to the at least one backscattered wave comprising:
forming the temporal signature between a time of transmission of the at least one ultrasonic wave to the at least one target and a time of reception of the at least one output signal by the ultrasonic receiver; and
encoding a spatial location of the at least one backscattered wave with the temporal signature.

12. A method for ultrasound imaging, the method comprising, at a computing device:
causing at least one ultrasonic wave to be transmitted towards at least one target;
receiving at least one output signal generated by an ergodic relay coupled to the at least one target, the at least output signal generated by the ergodic relay applying a temporal signature, as a function of a location of the at least one target relative to the ergodic relay, to at least one ultrasonic wave backscattered by the at least one target;
reconstructing at least one image of the at least one target based on the at least one output signal and a dictionary of reception signals; and
outputting the at least one image as reconstructed.

13. The method of claim 12, wherein the at least one image of the at least one target is reconstructed based on the at least one output signal and the dictionary of reception signals as follows:
$$s ( \vec{r} ) = {\sum }_{t} {e}_{\vec{r}} ( t ) \times s ( t )$$
where s(\vec{r}) is the at least one image of the at least one target as reconstructed, s(t) is the at least one output signal, and e_{\vec{r}}(t) is the dictionary of reception signals determined from a unique point reflector located at f.

14. The method of claim 12, further comprising performing a calibration procedure for determining the dictionary of reception signals, the calibration procedure comprising imaging at least one strong scatterer with an ultrasound probe to generate the reception signals and at least one reference image associated therewith.

15. The method of claim 12, wherein at least one deep learning technique is used to at least one of reconstruct the at least one image of the at least one target and perform a calibration procedure for determining the dictionary of reception signals.

16. The method of claim 14, wherein at least one harmonic imaging technique is used to at least one of said reconstruct the at least one image of the at least one target and said perform the calibration procedure.

17. The method of claim 12, wherein the at least output signal is generated by the ergodic relay applying the temporal signature to the at least one backscattered wave, comprising:
forming the temporal signature between a time of transmission of the at least one ultrasonic wave to the at least one target and a time of reception of the at least one output signal; and
encoding a spatial location of the at least one backscattered wave with the temporal signature.

18. A calibration method for ultrasound imaging, the method comprising:
causing at least one ultrasonic wave to be transmitted towards at least one strong scatterer;
receiving at least one reception signal generated by an ergodic relay coupled to the at least one strong scatterer;
associating the at least one reception signal with a position of the at least one strong scatterer; and
storing the associated at least one reception signal in a dictionary.

19. The method of claim 18, wherein the at least reception signal is generated by the ergodic relay by applying a temporal signature, as a function of a location of the at least one strong scatterer relative to the ergodic relay, to at least one ultrasonic wave backscattered by the at least one strong scatterer.

20. The method of claim 18, wherein the at least one ultrasonic wave is caused to be transmitted towards the at least one strong scatterer comprising a cloud of microbubbles.
